(19) United States
(12) Patent Application Publication
(10) Pub. No.: US 2026/0049289 A1
(43) Pub. Date: Feb. 19, 2026

(54) ARRAY PLATFORM FOR HIGH-THROUGHPUT ORGANOID PROFILING

(71) Applicants: CZ Biohub SF, LLC, San Francisco, CA (US); The Board of Trustees of the Leland Stanford Junior University, Stanford, CA (US)

(72) Inventors: Polly M. Fordyce, Menlo Park, CA (US); Christina Curtis, Stanford, CA (US); Alexandra F. Sockell, Stanford, CA (US); Wing Wong, Stanford, CA (US)

(21) Appl. No.: 19/116,933

(22) PCT Filed: Oct. 2, 2023
(86) PCT No.: PCT/US2023/075710

§ 371 Date: Mar. 28, 2025

Related U.S. Application Data
(60) Provisional application No. 63/378,229, filed on Oct. 3, 2022.
(60) Provisional application No. 63/380,196, filed on Oct. 19, 2022.

(51) Int. Cl. C12N 5/09; C12M 1/32; C12M 1/34; C12M 3/00; G01N 33/58

(52) U.S. Cl. CPC C12N 5/0693; C12M 21/08; C12M 23/12; C12M 41/36; G01N 33/582; C12N 2510/00

(57) ABSTRACT

Organoid culture and image-processing methods and systems are described. Such methods and systems provide the ability for high-throughput characterization of a variety of phenotypes at single-organoid resolution.

### CROSS-REFERENCE TO RELATED APPLICATIONS

This application claims priority benefit of U.S. Provisional Application No. 63/378,229, filed Oct. 3, 2022; and U.S. Provisional Application No. 63/380,196, filed Oct. 19, 2022, each of which is herein incorporated by reference for all purposes.

### STATEMENT AS TO RIGHTS TO INVENTIONS MADE UNDER FEDERALLY SPONSORED RESEARCH AND DEVELOPMENT

This invention was made with Government support under contract CA238296 awarded by the National Institutes of Health. The Government has certain rights in the invention.

### BACKGROUND

The development of 3D organoid culture methods in recent years has revolutionized in vitro studies of various diseases including cancer (1-3). Organoid models are significantly better than 2D cell cultures at recapitulating biological processes such as multi-lineage differentiation and protein expression (4, 5). In addition, primary 3D organoids provide a more physiologically relevant genomic background than conventional 2D cell lines for modeling disease progression via genome engineering (6, 7). In these engineered models, wildtype cells obtained from normal donor tissue are sequentially genome-edited in vitro using a combination of CRISPR and other lentiviral techniques to mimic the accumulation of mutations that occur during cancer transformation, with multiple lines of evidence indicating that this process recapitulates tumor progression both phenotypically and genetically (6-8).

With each subsequent genome-editing, organoid lines approach a “transformed”, cancer-like state. Recently, gastric organoid models have been developed to study the molecular determinants implicated in gastric cancer (6, 9). Using a longitudinal sampling of the engineered gastric organoids, it was demonstrated that this model recapitulates many of the genomic changes seen in advanced gastric cancer (9).

Despite the abundance of genomic methods, quantitatively characterizing how engineered mutations alter organoid phenotypes at various stages of transformation remains challenging, largely due to the limited phenotypic resolution within a standard bulk culture (11). The most common strategy for culturing organoids in 3D involves resuspending dissociated cells in Matrigel, a commercially available extracellular matrix (ECM) mimic (12). However, in this method, organoids are generally formed from clusters of aggregated cells, rather than from a single cell that expands independently, making it difficult to determine whether an observed phenotypic trait reflects the stochasticity of small deposited cell populations or is intrinsic to each individual cell at the start of organoid growth (13, 14). Distinguishing intrinsic from extrinsic heterogeneity is further complicated by variations in the organoid's growth environment, as organoids seeded close to other organoids may be affected by cell-cell paracrine signaling, and their position relative to the margins of the Matrigel hemisphere can impact the diffusion of growth factors and/or drugs (15, 16). Finally, the same observed bulk growth differences could also result from differences in either median organoid growth rate (17) or the fraction of the population that grows, complicating interpretation.

Microfabricated microwells represent an alternative to bulk culture methods and have been used in the materials science and tissue engineering fields for 3D cell culture, with many different variations in microwell design (13, 18-21). However, most methods reported to date rely on cellular aggregation to fuel the development of spheroids (13, 18, 20), rather than organoids derived from a single cell, which preclude follow-up interrogations into cellular heterogeneity. In addition, unlike in 2D cell lines, there is a lack of comprehensive image analysis pipelines to quantitatively track the growth trajectories and other phenotypic traits of live 3D organoid cultures over time (22). These issues have made it challenging to characterize the effect of heterogeneity in a population on its downstream growth characteristics, such as changes in single-cell growth rates or the ability to maintain normal apical-basal polarity (e.g. in epithelial organoids), both of which are thought to be altered early in epithelial tumorigenesis (23, 24). Moreover, many of these platforms have lacked the ability to selectively retrieve organoids of interest for downstream investigations, making it difficult to link phenotype to genotype (19).

### BRIEF SUMMARY

The present disclosure provides a culture and image-processing method and platform that provides the ability for high-throughput characterization of a variety of phenotypes at single-organoid resolution. Thus, for example, cell size and position over time can be quantified to determine single organoid growth rates and migration behavior for thousands of organoids in parallel. In other embodiments, engineering and staining organoids to fluorescently-label cell nuclei and actin distributions allows the identification of cells with abnormal polarity, a hallmark of transformation. Organoids of interest can further be retrieved from the growth compartment, e.g., for sequencing or alternative molecular profiling.

The organoid platform of the present disclosure offers multiple advantages over other existing organoid culture methods. In comparison to bulk culture, the present method, e.g., when combined with 2D or 3D image analysis, provides the ability to measure quantitative phenotypes for individual organoids, such as single organoid growth rates, division times, and migration distances, rather than being restricted to bulk averages, such as fold change in cell number for an entire population. Unlike commercially available methods or closed microfluidic systems, the open planar arrays described herein do not require specialized instrumentation and are thus easy to adapt to existing cell culture workflows. Further, complete organoids can be generated from single cells, e.g., to evaluate tumorigenesis, including cancer progression over time, e.g., using gene-edited organoid models. In addition, the open nature of the arrays provides the ability to retrieve organoids with phenotypes of interest, which can then be processed to evaluate the basis of the phenotype, e.g., by direct sequencing, or expanded clonally in vitro to generate more biomass for additional phenotypic or molecular profiling.

The single organoid resolution of the platform of the present disclosure can also be employed in applications such as personalized drug screening of patient-derived human organoids. In particular, the platform provides the ability to characterize the drug response and associated phenotypic changes of every single organoid seeded in the microwells independently, offering the potential to screen patient-derived tumor organoids against a wide variety of drugs using much lower input materials.

Thus, in one aspect, provided herein is a method of monitoring organoid development in parallel for a plurality of organoids, each arising from one or more organoid progenitor cells, said method comprising: (i) distributing organoid progenitor cells from a population of organoid progenitor cells into array compartments to provide an open planar array comprising a plurality of organoid growth compartments in which each organoid growth compartment comprises one or more organoid progenitor cells; (ii) incubating cells in the organoid growth compartments of the open planar array under growth conditions in which the one or more organoid progenitor cells in each organoid growth compartment divides; (iii) imaging compartments of the open planar array at multiple time intervals; and (iv) analyzing at least one phenotypic parameter of multi-cell structures growing in at least a portion of the organoid growth compartments imaged in step (iii). In some embodiments, distribution of organoid progenitor cells into the open planar array provides a plurality of organoid growth compartments, each comprising a single organoid progenitor cell. In some embodiments, organoids in each growth compartment of the open planar array are cultured under the same conditions. In some embodiments, the population of organoid progenitor cells is genetically distinct across the population. In some embodiments, (iii) and (iv) occur simultaneously. In some embodiments, the method further comprises distributing additional co-culture cells into at least a portion of the organoid growth compartments containing organoid progenitor cells, or a multi-cellular structure arising from the organoid progenitor cells. In some embodiments, the co-culture cells are immune system cells, such as T cells, dendritic cells, macrophages, or natural killer cells. In some embodiments, the at least one phenotypic parameter is rate of cell division, cell polarity, or cell migration. In some embodiments, step (iv) comprises detecting a signal from a cellular molecule labeled with a detectable label, such as a fluorescent label. In some embodiments, the population of organoid stem cells is genetically modified to introduce the cellular molecule into cells. In some embodiments, the fluorescent label is attached to an agent that specifically binds the molecule. In some embodiments, the population of organoid progenitor cells comprises epithelial cells. In some embodiments, the population of organoid progenitor cells comprises cells having a mutation at one or more genetic loci associated with a cancer, or two or more genetic loci associated with the cancer. In some embodiments, the mutations at genetic loci associated with cancer are introduced into the cells by genome editing. The cancer can be any number of cancers, e.g., gastric cancer, colorectal cancer, pancreatic cancer, breast cancer, ovarian cancer, uterine cancer, prostate cancer, bladder cancer, liver cancer, lung cancer, esophageal cancer, head and neck cancer, or renal cancer. In some embodiments, the compartments are microwells. In some embodiments, step (i) comprises loading cells using centrifugation. In some embodiments, imaging occurs at least once a day following distribution of single organoid stems cells into compartments. In some embodiments, the method further comprises retrieving cells from at least a portion of the organoid growth compartments for genetic analysis. In some embodiments, retrieving cells comprises aspirating cells. In some embodiments, genetic analysis comprises RNA sequence, ATAC sequence analysis, whole genome sequencing, or methylation profiling.

In a further aspect, the disclosure provides a platform for monitoring organoid growth comprising (i) an open planar array comprising at least 100 organoid growth compartments, wherein each organoid growth compartment is a microwell comprising a three dimensional multi-cellular structure arising from one or more organ progenitor cells and (ii) an imaging system. In some embodiments, each of the three dimensional multi-cellular structures in a plurality of microwells comprises cells comprising a molecule labeled with a fluorescent label. In some embodiments, the fluorescent label is attached to an agent that specifically binding the molecule. In some embodiments, each three dimensional multi-cellular structure in a plurality of microwells comprises epithelial cells. In some embodiments, each three dimensional multi-cellular structure in a plurality of microwells comprises cells having a mutation at one or more genetic loci associated with cancer. In some embodiments, the three dimensional multi-cellular structure arose from a single cell that was genome edited to comprise the mutation. In some embodiments, the cancer is gastric cancer, colorectal cancer, pancreatic cancer, breast cancer, ovarian cancer, uterine cancer, prostate cancer, bladder cancer, liver cancer, lung cancer, esophageal cancer, head and neck cancer, brain cancer, or renal cancer.

### BRIEF DESCRIPTION OF THE DRAWINGS

FIG. 1A-F: Illustrative microwell platform for organoid evaluation, which in this instance, are employed for high-throughput phenotyping of gastric cancer organoids. (A) Sequential genome-editing of TP53 and ARID1A in human gastric organoids revealed progressive phenotypic changes. Loss of epithelial apical-basal polarity was associated with P53/ARID1A DKO organoids. Organoid cultures were seeded in Matrigel and allowed to grow for 6 days before bulk measurements of growth and microscopy examination. (B) DKO organoids were significantly faster than P53KO organoids in terms of growth rate in bulk measurement. (C) Schematic of the initial microwell experiments using brightfield microscopy. Each microwell array was placed onto a well of a 12-well tissue culture plate. Single cells dissociated from organoids were then seeded into the microwells and allowed to expand. (D) An example of microwell arrays with organoids. (E) An example image showing growth trajectories of single cells seeded in the microwell. (F) growth classification graph FIG. 2A-E: DeepCell neural net accurately identifies cells within fluorescence images to enable high-throughput image processing and organoid growth quantification. (A) Experimental pipeline for cell engineering and imaging. (B) Pipeline for DeepCell training and validation (left) and performance metrics (right). (C) 2D histogram comparing the number of DeepCell-predicted cells and manually annotated cells per microwell (N=912 total microwells). Dashed red line indicates linear regression. (D) Example raw and DeepCell-thresholded images for 2 microwells over 108 hours of imaging (left); DeepCell-annotated areas and cell numbers over time for all images (right). Red line denotes the exponential growth equation to the total area over time; the fit equation and returned parameters (growth rate per day) are indicated. (E) Example single cell position traces over time.

FIG. 3A-H: Microwell experiments with fluorescence microscopy and DeepCell analytical pipeline. (A) Pie charts indicating the number of P53KO and P53/ARID1A DKO cells loaded per microwell across each experiment. (B) Growth rates as a function of the number of cells per microwell across each experiment for P53KO (top) and P53/ARID1A DKO (bottom) organoid lines. (C) Growth rates for all single cells across each experiment for P53KO (top) and P53/ARID1A DKO (bottom) organoid lines. (D) Heat map showing median growth rates for all microwells within macrowell A1 for experiment #1; median growth rates are indicated within each well. (E) Kernel density plots showing the time to first cell division for microwells containing a single cell from either P53KO or P53/ARID1A DKO organoid line; dashed lines indicate median time to first cell division. (F) Example confocal images of normal and abnormal polarity organoids. (G) Example fluorescent images of normal and abnormal polarity organoids. (H) Quantification of proportion of normal vs. abnormal polarity organoids in P53KO vs. DKO organoid lines.

FIG. 4A-F: Microwell experiments under controlled experimental conditions reveal changes in the genomic landscape that is associated with the increase in growth rate in P53KO organoids. (A) Schematic of experiments #4- #6 where chemically defined growth media were used instead of conditioned media. Both P53KO and DKO organoid lines were revived from cryopreservation and lentivirally transduced with H2B-mCherry at a defined MOI of 0.1. The 200 μm microwells were used to facilitate the retrieval of organoids of interest using a 250 μm PEEK tubing setup. At the start of experiments #4 and #6, shallow whole-genome sequencing was performed. (B) Pie charts indicating the number of P53KO and P53/ARID1A DKO cells loaded per microwell across each experiment. (C) Growth rates as a function of the number of cells per microwell across each experiment for P53KO (top) and P53/ ARID1A DKO (bottom) organoid lines. (D) The P53KO organoids grew increasingly faster with each experiment while the growth rates of DKO organoids remained relatively constant throughout the three experiments. (E) Whole-genome copy number plot for P53KO (green box) and DKO organoids (purple box) at experiments #4 and #6. The P53KO organoids at experiment #6 acquired additional aberrant copy number alterations in genes associated with cell proliferation and tumorigenesis in gastric cells. This includes losses of multiple mucin genes on chromosome 11, as well as loss of FHIT that has been implicated in chromosomal instability.

FIG. 5A-I: Single organoid retrieval and sequencing revealed changes in chromatin accessibility associated with cellular morphology and adhesion. (A) Schematic of organoid picking experiment. Single organoids were retrieved using a syringe pump connected to a 3D-printed microscope adapter via PEEK tubing. Retrieved organoids were dispensed into a clean vial and subsequently prepared for sequencing. (B) Images of all retrieved organoids. Top: organoids with normal polarity as evidenced by a robust actin ring around the lumen. Bottom: organoids with abnormal polarity as evidenced by disorganized actin signals. (C) ATAC-seq quality control steps to remove samples with insufficient read depths and peaks. (D) Heatmap of differentially accessible peaks between normal and abnormal organoids. (E) Gene ontology analysis of differentially accessible peaks revealing enrichment of the peaks in pathways associated with cellular adhesion. (F) Principal component analysis revealing clear separation between normal and abnormal organoids. (G) Transcription factor footprinting analysis revealed enriched binding of SP-family transcription factors in abnormal organoids. (H) Representative plots showing increased binding of SP-family transcription factors in PPP2R1B and LLGL2 which are involved in regulating apical-basal polarity in epithelial cells. (I) Volcano-plot showing differentially expressed genes in RNAseq data. Two of the four upregulated genes in abnormal organoids are known to harbor binding sites for SP-family transcription factors.

FIG. 6: Representative drug evaluation of patient-derived pancreatic ductal adenocarcinoma organoids treated with gemcitabine. Upper panel, organoids sensitive to gemcitabine; lower panel, organoids resistance to gemcitabine.

### DETAILED DESCRIPTION

### Terminology

The term “organoid” as used herein refers to an in vitro three-dimensional culture of cells that have a self-generated structure that comprises multiple cell types that are present in a cancerous, normal adult, or developing organ tissue. In the context of the present disclosure, reference to an “organoid” includes spheroids.

An “organoid progenitor cell” as used herein refers to a cell that is capable of forming an organoid, i.e., differentiating into multiple cell types that represent tissues of an organ when cultured. Such progenitor cells include embryonic stem cells, induced pluripotent stem cells, somatic stem cells or tissue-derived progenitor cells, cancer cells, or genetically manipulated normal cells from a tissue of interest that comprise modifications, e.g., introduced by gene editing, to genes known to be mutated in cancer, e.g., driver genes.

For purposes of this application, an “organoid growth compartment” refers to any partially enclosed compartment that separates one organoid from another and provides an opening for access to the organoid, e.g., for retrieving the organoid for molecular profiling or further culturing.

An “array” as used in the present disclosure refers to a collection of single organoid compartments, at least a portion of which comprise organoid progenitor cells that give rise to different organoid structures. An array may be an “ordered array” in which the compartments are addressable and can be assigned to known locations. In some embodiments, an array is contained in a larger compartment (a “macrocompartment”), e.g., to provide the same culture conditions, e.g., the same media, growth factors, or other agents present in culture media. As used herein, an “open planar array” refers to an array comprising multiple organoid growth compartments, each of which is used to grow a single organoid. An open array may be contained in a macrocompartment, which may have a removable cover.

A “three-dimensional” or “3-D” culture system as used herein refers to an apparatus comprising one or more arrays that comprises multiple organoid growth compartments, where each organoid growth compartment accommodates a three-dimensional organoid structure that arises from one or more progenitor cells. Such a culture system can also comprise macro-containers, e.g., containing one or more arrays, medium required for cell growth and differentiation, and factors such as extracellular matrix components, e.g., Matrigel or extracellular matrix hydrogels.

### Arrays/organoid Growth Compartments

An array can comprise any type of partially closed compartments to separate organoids from one another, but typically comprises microwells for culturing organoids that arise from one or more progenitor cells seeded into individual microwells. The term “microwell” is used for convenience in describing arrays in this section, but the same criteria apply to other types of containers. In some embodiments, a microwell array (also referred to as a “microwell device”) may comprise anywhere from tens of microwells to hundreds of millions of microwells, e.g., from 50 microwells to 500 million or more microwells. In some embodiments, a microwell array, e.g., on a glass slide of about 1 inch by 3 inches, comprises over 100 million microwells. In some embodiments, e.g., a multiwell plate, a microwell array comprises at least 200 or at least 300 microwells. In some embodiments, an array comprises from 50-1,000 microwells; or from 50-500 microwells.

A microwell can be square or rectangular, or an alternative shape, e.g., a cylindrical shape. In some embodiments, the well is flat-bottomed. In other embodiments, the bottom of the well may be U-shaped. In some embodiments, individual compartments can range in size from 1 μm-5 mm in diameter or per side for square/rectangular microwells. In some embodiments, a microwell can be from 1 μm-2 mm per diameter or per side for square or rectangular wells. In some embodiments, a microwell has a volume of from 10 μl to 200 μl. In some embodiments a microwell has the dimension of a well of a commercially available 384-well plate, e.g. diameter of about 3.3-3.7 mm.

A microwell device can be obtained from commercial vendors, e.g., a 384-well plate, or can be a custom array, and can be of any suitable material. For custom arrays, a variety of suitable fabrication methods, such as wet etching, reactive ion etching, machining, photolithography, soft lithography (for example, multi-layer soft lithography), hot embossing, injection molding, laser ablation, in situ construction, or plasma etching may be employed. A non-limiting example of a suitable fabrication method uses multi-layer soft lithography, e.g., a described in the “Techniques Section”. Selection of a suitable fabrication method depends at least in part on the material to be used in the fabrication. Materials that may be in the fabrication include, but are not limited to, silicon, glass, quartz, polydimethylsiloxane (PDMS), polymethylmethacrylate (PMMA), thermoset polyester (TPE), polycarbonate (PC), cyclic olefin copolymer (COC), polystyrene (PS), polyvinylchloride (PVC), and polyethyleneterephthalate glycol (PETG). For example, in some embodiments, a microwell array is produced using standard soft-lithography. In some embodiments, PDMS is employed for formulating the microarray. In some embodiments, the thickness of material, e.g., PDMS, is about 0.5 mm, to provide high quality image analysis.

One of skill in the art understands that prior to use, a microarray deice may be treated so that the surface is suitable for cell growth. For example, in some embodiments, a microwell array is treated with oxygen or air plasma or coated with a PBS-BSA solution.

Cells are distributed into microwells to provide a desired seeding density, one or more, of progenitor cells. In some embodiments, an array is generated in which a plurality of compartments contain a single organoid progenitor cell. For example, cells can be distributed at set dilution concentrations (in accordance with Poisson loading) such that at least a portion, e.g., about 11-20% of the compartments will contain a single cell, i.e., the probability of multi-cell loading is reduced via Poisson statistics.

In some embodiments, cells are loaded by centrifugation. In some embodiments, an extracellular matrix material is added to each well following loading to support organoid growth and development, follow by the addition of media.

### Organoid Progenitor Cells

Any cell that is capable of developing into an organoid can be employed in the high throughput organoid profiling methods described herein. In some embodiments, an organoid progenitor cell is a stem cell obtained from tissue of an adult subject. In some embodiments, the organoid progenitor cell is a pluripotent stem cell, such as an induced pluripotent stem cell (iPSC) or an embryonic stem cell. In some embodiments, progenitor cells are obtained from normal tissue that is genetically modified to introduce mutations, e.g., mutations that play a role cancer of that tissue.

### Pluripotent and Embryonic Stem Cells

As indicated above, organoid progenitor cells include pluripotent stem cells such as inducible pluripotent stem cells (iPSCs) and embryonic stem cells (ESCs). iPSCs can be obtained from somatic cells by co-expression of defined pluripotency factors, such as Oct4, Sox2, c-Myc, and Klf4. Once re-programmed, iPSCs can typically proliferate indefinitely in vitro and differentiate into all three primary germ layers, i.e., ectoderm, mesoderm, and endoderm. Thus, an iPSC can also be obtained from an established iPSC cell line. Similarly, an ESC, which can be obtained from the inner cell mass of a pre-implantation blastocyst, or established ESC cell lines, can differentiate into all three primary germ layers. Individual iPSC and ESC cell can thus proliferate and self-organize to mimic various organ tissues, including, but not limited to intestine, stomach, kidney, brain, lung, liver, kidney, pancreas, and brain, among others, when cultured in the presence of suitable growth and differentiation factors (reviewed, for example, by Yin et al, Cell Stem Cell 18-25-38, 2016; Kim et al, Nature Reviews Molec. Cell Biol. 21:571-584; Hofer & Luotoll, Nature Review Materials 6:402-420, 2021).

In some embodiments, an organoid progenitor cell is an adult stem cell (ASC), which refers to an undifferentiated cell found among differentiated cells in a tissue or organ. An adult stem cell can renew itself and can differentiate to yield some or all of the major specialized cell types of the tissue or organ. Human organoids from ASC can be generated from most endoderm-derived tissues (e.g., intestine, colon, stomach, pancreas, lung, bladder, etc) and from gender-specific tissues (prostate, endometrium, fallopian tube and mammary gland). Adult stem cells have also been identified, for example, in the brain, heart, and skeletal muscle.

In some embodiments, an organoid progenitor cell is generated by genetic manipulation of cells obtained from normal organ tissue, e.g., stomach, colorectal, esophagus, intestine, liver, prostate, mammary gland, biliary tract, epithelial cells, mesenchymal cells or other cells. In some embodiments, organoid progenitor cells may be obtained from cancerous tissues.

In some embodiments, or an organ progenitor cell can be generated by genetically modifying a cell, e.g., using a gene editing technique such as CRIPR/Cas, from a normal tissue to mutate a gene involved in tumorigenesis, e.g., oncogenes identified in malignancies of the normal tissue from which the cell is obtained. In some embodiments, multiple mutations may be introduced into a cell.

As indicated above, organoid progenitor cells are distributed into organoid growth compartments to provide a desired number of cells to seed organoid generation. In some embodiments, the array comprises a plurality of organoid growth compartments that contain a single cell. In some embodiments, the population of cells distributed into the compartments may be obtained from a clonally expanded stem cell culture; or from an organoid that has been cultured to enrich for stem cells.

In some embodiments, the population of progenitor cells distributed into growth compartments comprises cells that are genetically distinct across the population, i.e., at least a portion of the cells can be distinguished from other cells in the population based on DNA sequence, or epigenetic modification, e.g., methylation.

### Organoid Culture

Organoid progenitor cells are cultured in conditions suitable for the organoid of interest (see, Kim et al, Nature Rev. Molec. Cell Biol. 21:571-584, 2020 and references cited therein). Organoid culture medium components include, e.g., basement membrane and/or extracellular matrix extracts, growth factors, hormones, mitogens, small molecules, and medium components. In some embodiments, the medium is a chemically defined medium. In some embodiments, the medium comprises constituents such as fetal bovine serum, or other undefined growth components, e.g., extracellular matrices, such as Matrigel™. Suitable basal media that can be employed include common media such as Iscove's Modified Dulbecco's Medium (IMDM), Ham's F12, Advanced Dulbecco's modified eagle medium (DMEM) or DMEM/F12, RPMI-1640, or any other suitable medium. In some embodiments, a culture medium that supports growth to obtain a desired organoid can be obtained commercially.

In typical embodiments, an array can be contained in a macro-container, i.e., larger compartment, so that all of the members of the array are subject to the same growth media.

In some embodiments, arrays are cultured under different culture conditions to evaluate stimulatory or inhibitor effects on cell growth and organoid development.

In some embodiments, arrays are cultured in the presence of different agents, such as small molecules or other therapeutic agents, e.g., peptides, inhibitory RNAs and the like, e.g., wherein the progenitor cells are derived from tumors from patients.

In some embodiments, progenitor cells and/or developing organoids can be co-cultured with other cell types. In some embodiments, cells are incubated with immune cells, such as CD4+, CD8+ T cells, regulatory T cells; dendritic cells, macrophages, natural killer cells, or other cells of the immune system. In some embodiments, the T-cell can be an engineered T cell, such as a CAR-T cell. In some embodiments, progenitor cells may be co-cultured with cells to evaluate competition, cell-cell-cooperativity and the like.

### Phenotypic Analysis of Arrays

Organoids can be evaluated to assess any phenotypic parameter of interest. In some embodiments, cell division, cellular migration, cell polarity, expression of markers of interest, organoid architecture, or any parameter of interest can be evaluated.

Image-based assays include detection of a signal from a cellular molecule labeled with a detectable label. The term “label,” as used herein, refers to any atom or molecule that can be used to provide a detectable and/or quantifiable signal. In some embodiments, the label can be attached, directly or indirectly, to a biomolecule. Labels include, but are not limited to, fluorophores, chromophores, mass labels, radioisotopes, electron dense particles, magnetic particles, spin labels, molecules that emit chemiluminescence, electrochemically active molecules, enzymes, cofactors, and enzyme substrates. In some embodiments, the label is a fluorophore or chromophore.

In some embodiments, phenotypic analysis comprises evaluating molecules associated with lipid membrane, organelles, nuclear envelope, cellular protein, lipid, carbohydrate or cytoskeletal content over a desired time period. In some embodiments, organoid progenitor cells can be genetically modified to contain a marker than can be evaluated at multiple time points during organoid growth and development.

In some embodiments, image analysis to assess organoid phenotypes can evaluate morphological characteristics of organoid cells, e.g., functional behaviors (e.g., migration, movement, growth), anatomical changes (e.g., cell migration, formation of organized structures), or signaling behaviors (e.g., presence of markers associated with particular signal pathways and/or any other parameter).

Phenotypic parameters can be measured over a variety of time intervals, e.g., from minutes to hours to days. In some embodiments, phenotypic parameters of interest are imaged at least once a day. In some embodiments, phenotypic parameters are imaged at least once a week. In some embodiments, phenotypic parameters are assessed at different intervals over a time course.

At the end of a desired time course, organoids contained in one or more organoid growth compartments of an array can be retrieved from the organoid growth compartment for further analysis. In some embodiments, the organoid is retrieved by aspiration. In some embodiments, the organoid can then be subjected to additional culture. In alternative embodiments, an organoid from an organoid growth compartment can be processed for molecular profiling.

Molecular profiling can be performed using any method including sequencing mRNA or other RNA populations of interest such as miRNA, snRNA, IncRNA and the like; genomic sequencing, including, but not limited to, haplotyping and phase determination, and genotyping. In some embodiments, genomic nucleic acids can be analyzed by ATAC-Seq to evaluate chromosome structure, methylation profiling, whole genome sequencing, or any other molecular profiling technique.

### Techniques Section Organoid Model and Growth Rates From Bulk Cultures

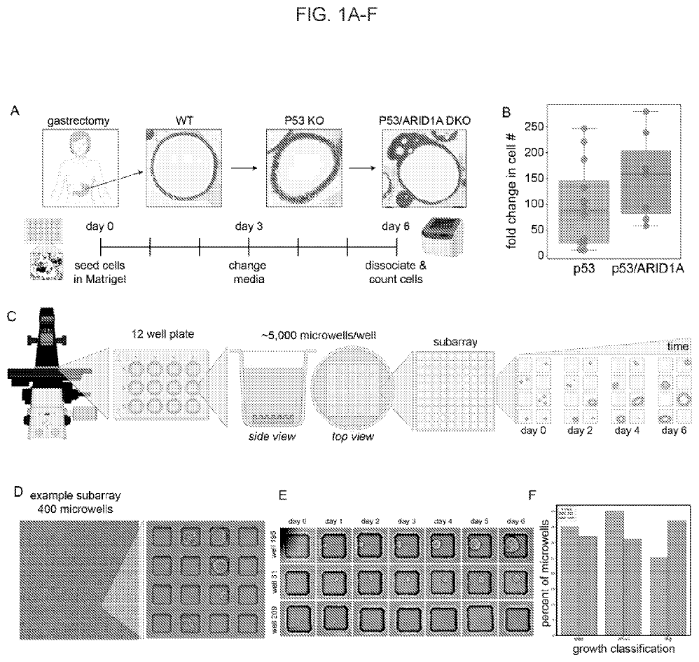

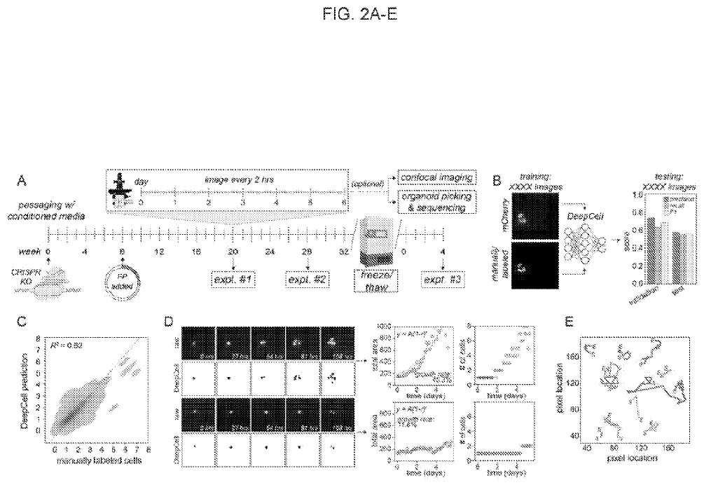

To investigate events that take place during early tumorigenesis, two recently developed gastric organoid models based on successive gene-editing (6, 9) (FIG. 1A) were employed. In the first (P53KO), CRISPR/Cas9 was used to knock out TP53, the master regulator of the DNA damage response and the most frequently altered locus in gastric cancer, particularly in the chromosomal instable (CIN) subtype (27). The second model (DKO) uses the same CRISPR/Cas9 method to additionally knock out ARID1A, a chromatin remodeler and part of the SWI/SNF complex that is frequently lost in the microsatellite-instable (MSI) subtype of gastric cancer (27, 28), on the P53KO background. This sequential gene-editing process is thought to recapitulate the stepwise transformation process from normal WT tissue as cells accumulate mutations and undergo expansions (7).

As shown previously (6), images from bulk cultures grown in Matrigel revealed qualitative phenotypic differences between these gastric organoid lines, with increasing cellular disorganization for organoids lacking either P53 or both P53 and ARID1A (FIG. 1A). Changes in cell growth, assessed by seeding bulk cultures with the same number of cells and then quantifying the fold-change in the number of viable cells after 14 days of passaging in conditioned media, also suggested that DKO organoids were more proliferative than P53KO organoids in bulk culture (FIG. 1B), consistent with previous observations (6). However, these fold-change increases could result from either individual organoids growing at a faster rate or from a larger fraction of organoids growing at the same rate. In addition, bulk measurements provide only a single rate value and preclude characterization of heterogeneous behavior within a given sample.

### Microwell Arrays for High-throughput Phenotyping of Single Organoids Over Time

To profile single-organoid growth at high-throughput, we developed a microwell platform for time-resolved phenotyping of thousands of organoids in parallel under near-identical conditions (FIG. 1C). Each single-layer PDMS microwell device contains arrays of 2,500-10,000 microwells of one of the two dimensions (either 100×100×80 μm or 200×200×80 μm, length×width×depth) placed directly at the bottom of each well (“macrowell”) of a 12 well culture plate (FIG. 1C). Devices are fabricated by spin coating PDMS onto master molds, thereby ensuring uniform thickness and enhancing image quality. To facilitate unique microwell indexing during downstream image processing, microwells are grouped within subarrays of 20×20 (100 μm) or 10×10 (200 μm) microwells, with a pattern of rotated microwells that uniquely identifies each subarray. The 100 μm diameter microwells are optimized for high-throughput imaging of thousands of organoids in the same experiment (experiments #1- #3), while 200 μm diameter microwells are best suited for retrieval of organoids with phenotypes of interest for single-organoid sequencing (experiments #4- #6).

To begin experiments, the devices are plasma-treated and coated with 0.5% BSA to render them hydrophilic. Initial measurements of single-cell occupancy as a function of starting cell concentration for P53KO cell lines established optimal concentrations of 6000 cells/mL for 100 μm microwells (26.15% of wells with a single cell) and 400 cells/mL for 200 μm microwells (31.33% of wells with a single cell), respectively; the fraction of microwells containing a given number of cells was well-fit by a Poisson distribution, consistent with expectations for stochastic loading.

For initial illustrative experiments, microwell arrays were seeded with single cells dissociated from P53KO organoids and then imaged daily via tiled bright field imaging (FIG. 1D). To visualize single organoid growth over time, we then: (1) stitched tiled images into a single image per macrowell per timepoint, (2) rotated stitched images to position microwell array edges parallel with image edges, (3) manually determined corner locations for each macrowell, and then (4) extracted individual microwell images from the rotated arrays by their position relative to the corner locations. A subset of 100 microwells for each organoid line was then manually inspected. Organoids grown in microwells appeared phenotypically similar to their counterparts grown in bulk culture, with a circular and cystic structure (FIG. 1D). However, individual organoids grown from single cells under identical experimental conditions often showed dramatically different growth behavior: while some organoids showed robust growth after seeding, defined as growth to >25% of the microwell area (25/100 P53; 37/100 DKO) (FIG. 1E, top), others either grew very little, growing to <25% of the microwell area (40/100 P53; 31/100 DKO) (FIG. 1E, middle) or showed signs of cell death/apoptosis (35/100 P53; 32/100 DKO) (FIG. 1E, bottom). An approximately equal proportion of P53KO and DKO organoids exhibited growth, and based on our manual classification, a greater proportion of DKO organoids showed larger and robust growth (>25% of the microwell area) compared to P53KO organoids (37% versus 25% of measured organoids respectively) (FIG. 1F).

High-Throughput Fluorescence Imaging Over Time can be Combined with Deep Learning to Track Thousands of Cells in Parallel

Brightfield microscopy made it possible to qualitatively assess organoid growth, but manual classification methods were both time-consuming and imprecise. In addition, brightfield images lacked sufficient resolution to accurately determine whether observed growth was a result of cell division or simply an increase in organoid volume due to osmotic swelling of the lumen and the cells (29, 30). To distinguish between these possibilities and simultaneously gain information about nuclear localization within developing organoids, both P53KO and DKO gastric organoid lines were engineered to express a nuclear fluorescent reporter by lentivirally inserting a mCherry-tagged copy of histone 2B (mCherry-H2B) (FIG. 2A); in parallel, the distribution of cytoskeletal proteins was profiled using live-cell labeling of actin or tubulin (e.g. SiR Actin Kit from Cytoskeleton Inc.). To determine optimal conditions for fluorescence imaging and pilot new analysis pipelines, P53 and DKO engineered cells were loaded (at a concentration of 4000 cells/mL) into 100 μm microwells across three separate replicate experiments. The first and second experiments took place after 5 and 7 months of continuous passaging in conditioned media; the third experiment took place after ˜8 months of continuous passaging followed by a freeze-thaw cycle (required due to COVID pandemic-related shutdowns) and an additional ˜1 month of passaging post freeze-thaw (FIG. 2A). After loading, the entire plate assembly was mounted on an automated microscope with an incubation chamber and tiled images were collected across the device in the bright field, mCherry, and Cy5 (for fluorophore-tagged actin molecules) channels at 2-hour intervals over 5 days for each experiment. (FIG. 2A). We then performed stitching, rotation, and microwell extraction for each imaging channel, similarly to the initial brightfield tests (ref. Methods). Time-course image processing for each well yielded a total of 24,044,400 microwell images (average 4,007,400 images per experiment).

To efficiently extract information about the number, size, and relative positions of cells within this large image dataset, DeepCell was employed. DeepCell is a deep-learning neural network that has been used to perform optical microscopy image segmentation to classify cells grown in 2D monolayers (25, 26). To test whether and under what conditions DeepCell can efficiently generalize to recognize and track cells within 3D organoids from 2D images, we manually labeled individual mCherry-tagged nuclei positions in 2711 images from the first experiment, used 2169 of these images and a transfer learning approach to train the organoid-aware DeepCell model, and validated performance with the remaining 542 images (ref. Methods). To test DeepCell's ability to generalize across experiments, model performance was also evaluated based on the number of accurately identified cells within 912 ‘test’ images from all 3 experiments (306, 348 and 258 images respectively) (FIG. 2B).

DeepCell counts showed very strong concordance with manual counts within the 5-day duration of the experiments (R2=0.81 in experiment 1; FIG. 2C).

Further visual inspection of images showing regions identified as cells by DeepCell confirmed the accuracy of automated annotations over time (FIG. 2D). We performed DeepCell prediction on a per-macrowell basis, with each macrowell taking an average of 145 seconds per time point for DeepCell processing. The DeepCell-annotated cell area and cell number over time could then be fit to an exponential growth curve to quantify these initial growth rates (FIG. 2D). In addition, we also demonstrated the ability of the pipeline to perform centroid tracking of single cells over time in order to identify the lower bound for the distance traveled by a single cell prior to the first cell division (FIG. 2E).

### P53/ARID1A DKO Cells Grow at Faster Rates Which Decrease With the Level of Occupancy

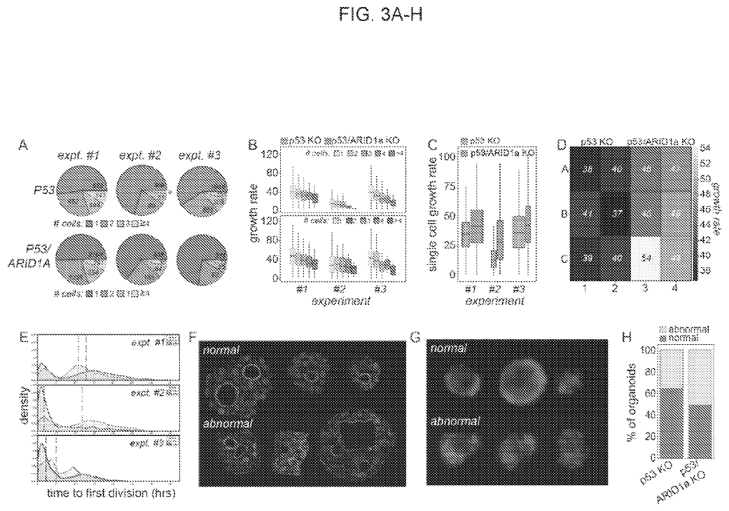

Using the trained DeepCell pipeline for 3D cultures, organoid growth rates were quantified across all three initial fluorescence microscopy experiments (FIG. 3A). In total, the three experiments profiled growth rates for 5812, 1328, and 3679 loaded microwells, respectively, with the number of cells loaded per microwell following stochastic Poisson distribution. Somewhat surprisingly, per-organoid growth rates for both organoid lines decreased as the number of cells initially seeded within the microwell increased, suggesting that increased paracrine signaling between cells in close proximity does not enhance growth (FIG. 3B). Similar to previously observed growth rates in bulk cultures, the single-organoid growth rates of the DKO line were higher than those of the P53KO line (FIG. 3C). However, variability was observed in growth rate across experiments, presumably due to differences in the composition of conditioned media which varied by batches (31). For instance, it was shown that the median overall growth rate per organoid line could vary by ˜2-fold between experiments. Despite this, DKO organoids consistently grew faster than P53KO organoids within the same experiment (FIG. 3C), establishing that the higher fold-change in cell numbers for DKO organoids in bulk experiments (FIG. 1B) was due to an enhanced growth rate in individual organoids and not simply due to a larger proportion of cells able to grow. The single-organoid growth rates for the same organoid line across different macrowells within the 12 well imaging plate varied only slightly, confirming an absence of macrowell-specific growth effects (FIG. 3D).

In addition to growth rate, we also used the DeepCell cell count predictions to calculate the time it took for a cell to complete its first cell division, by identifying the time point at which the microwell is predicted to contain n+1 the number of cells it held at the initial time point. This analysis was restricted to microwells seeded with a single cell. Interestingly, in each of the experiments and for both P53KO and DKO mutants, a bimodal distribution was observed, in which some cells divided very soon after the initial seeding, and the remainder took much longer to divide (FIG. 3E). Experiment-level variation was also observed in the time to first division. For example, most P53KO cells in experiment #2 began dividing soon after seeding, and only a small subset of cells divided at later time points. The total distance moved exhibited a somewhat similar modal pattern to time to first cell division, as cells that took longer to divide had more time for movement. Total distance moved was moderately positively correlated with the time to first division (R2=0.3209), and weakly negatively correlated with growth rate (R2=0.0148). Looking at the distance moved by time point, cells tended to move most immediately after seeding, and then settled down with decreasing movement before dividing.

### Disordered Polarity Is Associated With Transformation Stage

Changes in apical-basal polarity are commonly seen in transformed tissues across cancer types (24, 32), and we also recently observed such changes in the DKO gastric organoid model in a separate study (6). To assess whether these changes could be visualized in organoids grown in microwells, confocal imaging of both P53KO and DKO organoids in microwells was performed. Confocal imaging confirmed that both P53KO and DKO lines contained organoids that exhibited signs of abnormal polarity. To quantify these changes in polar organization, we then visually inspected final timepoint fluorescence images for 257 and 236 P53 KO and P53/ARID1A DKO cells from the first experiment and classified organoids as having either ‘normal’ or ‘abnormal’ apicobasal polarity. To be classified as ‘abnormal’, organoids were required to display 2 of the following 3 characteristics: (1) disorganized actin signal not restricted to organoid lumens, (2) lack of a central lumen ringed with actin, and/or (3) the presence of multiple small, disorganized lumens (FIG. 3F). A greater proportion of the DKO organoids exhibited an ‘abnormal’ phenotype (50.8% vs. 35.4% in the P53KO organoids), suggesting that progression towards a more transformed genotype drives greater cellular disorganization (FIG. 3G).

Controlled Experimental Conditions Reveal Chromosome Alterations that Drive Enhanced Organoid Growth

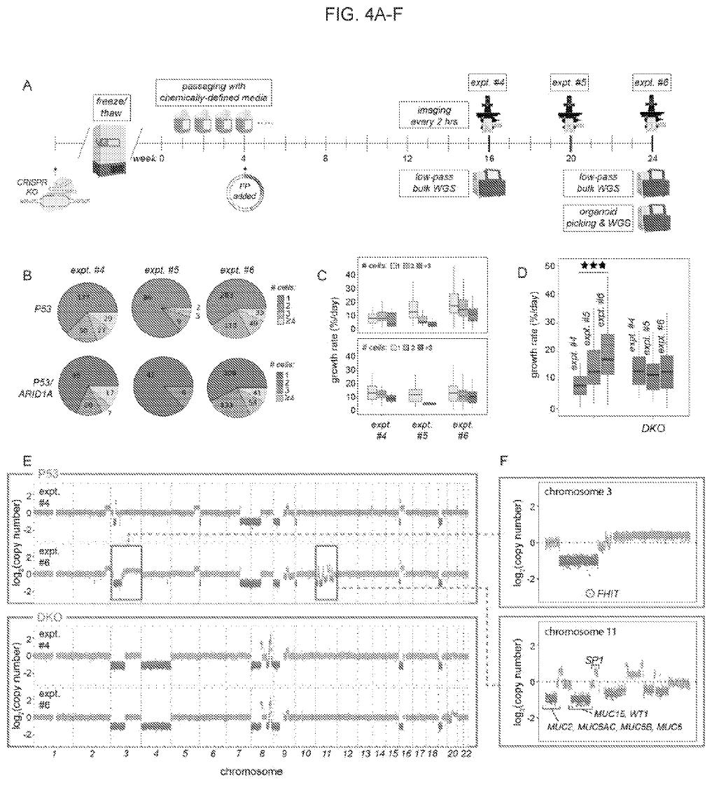

The high-resolution phenotyping data from these initial three experiments ( #1- #3) provided evidence that subtle differences in external conditions (i.e. different media batches) between experimental iterations can drive phenotypic differences that are sufficiently large that they could obscure meaningful biological variation (FIG. 3C). To systematically characterize and isolate phenotypic differences that arise directly from the deletions of P53 and ARID1A tumor suppressors and progression through transformation, these experiments were repeated under more tightly controlled conditions (ref. Methods). All cells were passaged using a standardized chemically defined media rather than conditioned media obtained from specialized cell lines. Three additional microwell phenotyping experiments were then performed under these controlled conditions (FIG. 4A). Furthermore, we used arrays of 200 μm microwells rather than 100 μm microwells in order to enable the retrieval of organoids with phenotypes of interest (diameter of retrieval tubing was 250 μm), with 422, 147, and 1009 loaded microwells per experiment, respectively (FIG. 4B).

Similar to the previous set of experiments described in FIG. 3, a general decrease in growth rates was observed with an increase in per-microwell occupancy in both P53KO and DKO organoids (FIG. 4C). Most other image-based parameters also recapitulated the observations made in FIG. 3. The DKO organoid growth rates remained relatively constant across experiments (FIG. 4D), highlighting the consistency of the growth media composition. However, while P53KO organoid growth rates were initially slower than those of DKO organoids in experiment #4 (consistent with observations during the first three experiments), P53KO growth rates increased steadily across the experiments until they eventually exceeded the DKO organoid growth rates by a significant margin (FIG. 4D). It has previously been demonstrated that P53KO organoids began to accumulate copy number variations (CNVs) relatively early after mutation engineering. To test if CNVs could be responsible for the observed increase in growth rates, we performed shallow whole-genome sequencing (sWGS) of both the P53KO and DKO from experiments #4 and #6. The sWGS for the P53KO organoids revealed additional large-scale copy number alterations on chromosome 3 in experiment #6, as well as apparent chromosome shattering encompassing all of chromosome 11, that were previously not observed in experiment #4 (FIG. 4E). By contrast, DKO cells showed no significant CNV changes across experiments (FIG. 4E).

We next searched the regions affected by CNVs in P53KO cells to identify specific genetic changes that could explain the observed increase in growth rate. The P53KO organoids from both experiment #4 and experiment #6 harbored focal deletion of FHIT locus, which was exacerbated in P53KO organoids from experiment #6 where the entire chromosome 3p arm was deleted (FIG. 4F). It was previously shown that FHIT is a common early loss in P53-null organoids (9), and it has also been recurrently observed in gastric cancer (33, 34). We hypothesize that the loss of both FHIT and P53 likely drives rapid accumulation of additional CNVs. On chromosome 11, loss of several regions containing multiple mucin genes (MUC2, MUC5AC, MUC5B, MUC6, and MUC15) was observed (FIG. 4F). These mucin genes are often dysregulated during malignant progression (35-37). For instance, abrogation of MUC5AC expression has been associated with increased cell proliferation and vascular invasion in gastric tumor cells (38, 39). Amplification of SPI1, which is a proto-oncogene upregulated in various cancer types and is typically involved in promoting cell proliferation (40-42) (FIG. 4F), was also observed.

Single Organoid Sequencing Reveals Changes in Chromatin Accessibility Associated with Cellular Morphology and Adhesion

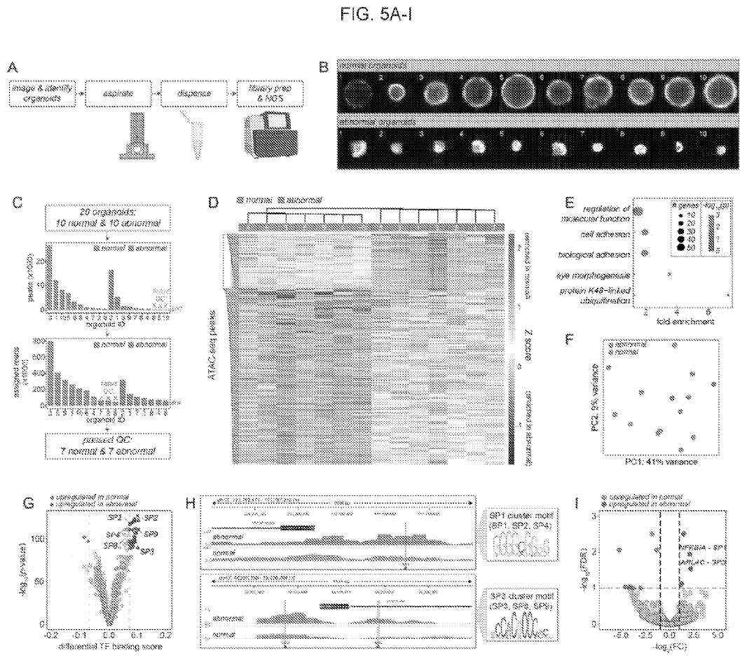

To further explore the molecular changes driving different polarity phenotypes, we developed a method to extract organoids of interest from the microwell arrays for downstream sequencing using a syringe pump and simple 3D-printed microscope adapter (ref. Methods; FIG. 5A). After staining organoids in microwells at the end of experiment #6 with a live-cell actin dye (ref. Methods), 10 organoids with normal polarity and 10 organoids with abnormal polarity from the DKO line (determined using the criteria described in Methods) (FIG. 5B) were retrieved. We then prepared libraries for single-organoid dual

### ATAC-seq and RNA-seq to Identify Differentially Accessible Regions (DARs) or Differentially Expressed Genes

As documented in the Methods section, the dual libraries preparation protocol was adapted from Li et al (2021). Owing to the low input amount from a single retrieved organoid, additional PCR cycles were required to amplify sufficient for sequencing, and hence the resulting libraries contained a relatively high number of duplicated reads (FIG. 5C). Besides the standard bioinformatic filters, we implemented additional filters and curations of the data to ensure sufficient biologically meaningful data were retained for final analysis (ref. Methods). Data quality was generally higher for ATAC-seq libraries compared to RNA-seq; thus, we focused our analyses on the ATAC-seq data, with RNA-seq data used only for validation of ATAC-seq results. In brief, read deduplication and removal of six organoid samples with <60,000 uniquely mapped reads or <500 accessible ATAC-seq peaks yielded chromatin accessibility data for 7 normal and 7 abnormal organoids with an average of 191,000 unique ATAC-seq reads and 4,425 accessible peaks per sample (FIG. 5C). Clustering based on ATAC-seq DARs clearly separated organoids with normal vs. abnormal polarity (FIG. 5D), and the DARs were enriched for gene ontology (GO) terms related to cell adhesion and morphogenesis (FIG. 5E). Principle component analysis (PCA) applied to the ATAC-seq data for each organoid also revealed a clear separation between the normal and abnormal polarity (FIG. 5F). At a molecular level, transcription factor (TF) footprinting analysis of the ATAC-seq data revealed that organoids with abnormal polarity had increased accessibility in chromosomal regions bound by the SP family TFs (FIG. 5G), which are involved in maintaining the epithelial cell polarity (43, 44), and dysregulation of SP family TFs have previously been found to be associated with a variety of cancer types (45, 46). For example, it was found that the SP family TFs were more highly bound to upstream promoter regions of several genes such as PPP2R1B and LLGL1 which are involved in establishing apical-basal polarity in epithelial cells (FIG. 5H) (44, 47, 48). Furthermore, analysis of DEGs in the RNA-seq data reveals multiple upregulated genes with known SP family target sites (FIG. 5I).

Representative Drug Testing Using Patient-Derived Pancreatic Ductal Adenocarcinoma (PDAC) Organoids Treated with Gemcitabine

This section illustrates personalized drug testing to evaluate treatment options for a condition of disease, e.g., cancer and for identifying resistance mechanisms to drugs.

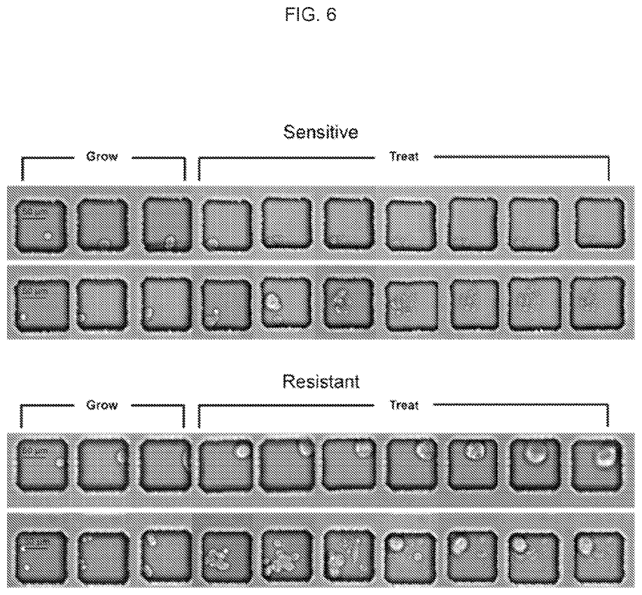

Organoids are a desirable choice for drug screening studies because they retain 3D cellular architecture and therefore recapitulate physiological conditions to a higher degree than traditional cell lines. As shown in FIG. 6, treating PDAC organoids with gemcitabine in the microwell platform revealed displayed heterogeneity in observed drug response, even for organoids derived from the same parental cells. Organoids sensitive to gemcitabine (FIG. 6, top panel) were growth-stunted and/or dissociated compared to resistant organoids (FIG. 6, bottom panel). Whole organoids that display resistance to a drug, can be retrieved and further evaluated, e.g., for genomic changes and/or changes in RNA expression profiles, to evaluate mechanisms of drug resistance, for example, innate versus acquired resistance.

### Summary of Material Presented in “Techniques Section

This section provides additional description of an easy-to-use, open planar array microwell platform for high-throughput, image-based phenotyping of organoids with the ability to retrieve single organoids of interest for additional downstream molecular profiling (e.g. single-organoid ATAC-seq or RNA-seq). As a first demonstration of the use of this platform, we applied it to investigate single-organoid behaviors for two engineered human gastric organoid models. Using an organoid-optimized deep learning model to identify and track individual cells within >8,000,000 microwell images, we were able to quantify organoid growth rates and positions over time with high accuracy. These measurements led to the identification of specific genomic changes capable of driving significant increases in organoid growth and also revealed genomic loci associated with losses of apical-basal polarity and cellular organization typically observed in cancer.

As previously described, this microwell platform offers multiple advantages over other existing organoid culture methods. In comparison to bulk culture, the microwell method combined with our custom 3D image analysis module allows us to precisely measure quantitative phenotypes for individual organoids, such as single organoid growth rates, division times, and migration distances, rather than being restricted to bulk averages, such as fold change in cell number for an entire population. Unlike commercially available methods or closed microfluidic systems, our microwell arrays do not require specialized instrumentation and are thus easy to adapt to existing cell culture workflows. Importantly, we are able to generate complete organoids from single cells, a particularly relevant advance for studying the early stages of tumorigenesis, in which a single transformed cell develops into a tumor. The open nature of the microwell arrays allows us to retrieve organoids with phenotypes of interest, which can then be either directly sequenced to investigate the molecular basis of the phenotype, or expanded clonally in vitro to generate more biomass for additional phenotypic or molecular profiling.

The single organoid resolution of the microwell platform can further extend the microwell's utilities to a personalized drug screening using patient-derived tumor organoids. In conventional drug screening via typical bulk response assays (e.g. IC₅₀ assays and AlamarBlue Cell Viability), very large cell numbers are required. This poses a particular challenge given the need to generate ample biomass from small tissue biopsies, which often require several months of organoid culture. This time frame increases the costs of such assays and wastes critical time, precluding real-time assessment of treatment response to guide patient care. Moreover, these conventional assays also overlook the genetic heterogeneity within the patient-derived tumor organoids and rely on a modal response, even though variation in cellular fitness can potentially be exploited therapeutically and further reveal resistance mechanisms (49). The microwell platform overcomes these challenges with the ability to characterize the drug response and associated phenotypic changes of every single organoid seeded in the microwells independently, offering the potential to screen patient-derived tumor organoids against a wide variety of drugs using much lower input materials.

In further embodiments of the present disclosure, a neural network that extends the DeepCell neural network employed in the technical analyses described above can be optimized further to track cell divisions and cell lineages with more frequent imaging intervals (e.g. on the order of minutes rather than hours) for fast-dividing cell types. In some embodiments, paracrine signaling can be evaluated with the loading of cells tagged with a different fluorescent protein. For example, the addition of fluorescent tags on proteins such as MUC5AC and PGC, which are commonly used as cell type markers in mucous cells and chief cells respectively, can be used to determine whether certain cell types are likely to promote or suppress growth. In some embodiments, adjacent apoptotic cells can be evaluated to determine whether they slow the growth of nearby cells by fluorescently labeling Annexin V (see, e.g., Ref. 50). In some embodiments, chromosome mis-segregation errors can be visualized during cell division using a reporter system, e.g., a H2B-mCherry nuclear reporter described herein with higher magnification imaging (see, Ref. 51).

Thus, the present disclosure provides a microwell-based 3D culture platform that can be easily adapted for the quantitative characterization of various image-based phenotypes. The technical analyses described above demonstrate the importance of single organoid measurements to uncover the full spectrum of behaviors in a population as opposed to bulk measurements, which can only capture population averages.

All patents, patent applications, and publications cited in this specification are herein incorporated by reference for the subject matter for which they are cited to the same extent as if each independent patent application, or publication was specifically and individually indicated to be incorporated by reference.

### Methods Employed for Technical Analyses

### Culturing of Crispr-engineered Gastric Organoids

The P53KO and DKO gastric organoids were previously generated for two separate studies prior to this (6, 9). Briefly, wild-type gastric corpus biopsies were obtained from a patient who underwent gastrectomy at Stanford University Hospital. Wild-type organoids were generated following a previously established protocol (9). The organoids were then sequentially engineered via CRISPR-Cas9 to knock out the expressions of TP53 and ARID1A.

The organoids were used in two separate sets of experiments. In the first set (experiments #1- #3), we conducted proof-of-principle tests to determine various types of measurements that we could perform by culturing single-cell derived gastric organoids in the microwells. Prior to the microwell experiments, organoids were first cultured in 24-well tissue culture plates. These organoids were maintained in growth media containing 50% Wnt3A/R-spondin1/Noggin conditioned media produced in house, 50% Advanced DMEM/F12, 1X Penicillin/Streptomycin/Glutamine, 1X Normocin, 1X B-27 Supplement, 1X GlutaMax, 1 mM N-Acetyl-L-cysteine, 500nM A83-01, 10 μM SB202190, 10 mM Gastrin, and 50 ng/ml EGF. The media was further supplemented with 10 μM Y-27632 and 2.5 μM CHIR-99021 during passaging to promote stem cell survival. The culture media were refreshed every 7 days and the organoid cultures were passaged once every 12-14 days. During each passage, old media was removed and 500 μl of TrypLE was added to each well to dissolve the Matrigel and dissociate organoids into single cells. After 30-40 minutes of incubation at 37° C., the cells were pelleted down by centrifuging at 500 g for 5 minutes. The supernatant was then removed, and the cell pellet was resuspended in wash media (Advanced DMEM/F12, 1X HEPES, 1X Penicillin/Streptomycin/Glutamine) for cell counting. Cell counting was performed by combining 10 μl of cell suspension with 10 μl Trypan Blue, then loading the mixture into a Countess chip for automated counting using the Countess II Cell Counter (Thermo Fisher). An appropriate volume of cells was transferred to a new tube, spun down at 500 g for 5 minutes, and the pelleted cells were resuspended in fresh Matrigel. Cells were re-plated in a new 24-well plate with 20000 cells per 40 μl of Matrigel dome. The plate was then incubated at 37° C. for 20 minutes to allow Matrigel to solidify; after which, 500 μl fresh growth media was added.

In the second set of experiments ( #4- #6), instead of using conditioned media as in the first set of experiments, we opted to use chemically defined complete growth media to prevent media batch effects from confounding our downstream analysis. The chemically defined media was composed of Advanced DMEM/F12, 1X Penicillin/Streptomycin, 1X Normocin, 1X N21-Max Supplement, 1X GlutaMax, 1 mM N-Acetyl-L-cysteine, 500nM A83-01, 10 μM SB202190, 10 mM Gastrin, and 50 ng/ml EGF. During passaging, 10 μM Y-27632, 2.5 μM CHIR-99021 and 200 ng/ml FGF10 were added. All other culture conditions remained the same as described in the previous section.

Lentiviral transduction to fluorescently tag organoids with histone H2B protein Addgene plasmid #89766 expressing H2B-mCherry fusion protein was packaged into lentiviral particles (by the Gene Vector Virus Core at Stanford University). Prior to transduction, the organoids were washed with PBS and incubated with TrypLE at 37° C. for 40 minutes. FBS was then added to quench the TrypLE reaction. After that, the dissociated cells were centrifuged at 500 g for 5 minutes and resuspended in growth media supplemented with 10 μM Y-27632. The H2B-mCherry lentiviral particles were added at an MOI of 0.1 to aliquots of 500 k cells to maximize the number of successfully transduced cells harboring just one copy of the transgene. This was done to reduce insertional mutagenesis and normalize the fluorescence intensity. The cell/virus suspension was transferred to a single well of a 24-well plate, and a 1-hour spinoculation at 600 g at 32° C. was performed. After spinoculation, cells were incubated 37° C. for four hours before being dissociated and pelleted down at 500 g for 5 minutes. After which, the pellet was resuspended in Matrigel followed by plating onto a 24-well plate. Organoids were allowed to recover for 3-5 days until mCherry expression was visible, then the transduced cells were FACS-sorted for cells expressing positive mCherry signal. Sorted cells were allowed to recover and expand for several passages before being used for experiments.

### Microwell Design and Fabrication

Design files for molds containing arrays of square microwells with a variety of different dimensions (from 100×100×80 μm to 1000×1000×80 μm; breath×width×height) were generated in AutoCAD and printed on standard transparencies at 30000 dpi.

Molds were created from SU-8 2100 photoresist (Microchem, Inc.) on a 4″ silicon test-grade wafer (University Wafer) according to the manufacturer's instructions. After fabrication, molds were treated with vapor deposition of 1H, 1H,2H,2H-perfluorooctyl-trichlorosilane (Sigma Aldrich) under vacuum for 10 minutes. Microwell devices were made from the molds using standard soft-lithography. Briefly, RTV 615 precursor solutions at a ratio of 10:1 (base elastomer: curing agent) (R. S. Hughes) were mixed using a THINKY mixer with 3 minutes of mixing followed by 3 minutes of degassing, both at 2000 rpm poured onto molds, and spun on a spin coater (Laurell Technologies) at 200 rpm with an acceleration of 133 rpm/s for 30 seconds to spread the PDMS and create a layer of PDMS approximately 0.5 mm thick, this thickness could be easily peeled off but remained thin enough for high-quality imaging using a long working distance objective. The PDMS was then degassed in a vacuum chamber for 15 minutes and baked at 80° C. for 20 minutes. After baking, the PDMS was peeled from the mold and cut into arrays of square devices. Prior to use, the devices were treated with 20% oxygen plasma for 8 minutes, placed at the bottom of 12-well plates with forceps, sterilized overnight by immersion in 70% ethanol, then treated with 0.5% PBS-BSA for >1 hour to render the devices hydrophilic for cell growth.

### Cell Loading Into Microwells

Dissociated organoid obtained during bulk culture passage were resuspended in WENR media to a concentration of 6000 cells/ml for 100 μm microwells and between 600-2000 cells/ml for 200 μm microwells. The cell suspension was pipetted directly onto BSA-treated microwell arrays, and plates were centrifuged at 500 g for 5 min to load cells into microwells. Following cell loading, excess media was aspirated and Matrigel was pipetted dropwise onto the microwell arrays containing cells. Plates were incubated at 37° C. to polymerize the Matrigel, then growth media supplemented with 10 μM Y-27632 and 2.5 μM CHIR-99021 and 200 ng/ml FGF10 was added.

### High-Resolution Time-Lapse Image Acquisition

For initial bright field imaging experiments (FIG. 1), organoids in microwells were grown in a standard tissue culture incubator and imaged 1X per day over 7-10 days using a Keyence BX-700 microscope. A grid acquisition with a 10X objective and 20X final magnification was performed for using the built-in brightfield capability of the Keyence microscope.

High-resolution time-lapse images for experiments #1-6 (FIGS. 2 and 3) were acquired on an inverted fluorescence microscope (Nikon Ti) with a motorized xy-stage (ASI MS-2000) and a camera (Andor Zyla 4.2+) set to acquire images at 2×2 binning for a final resolution of 1024×1024. Broad spectrum illumination was provided by a solid-state light source (Lumencor Sola). Images were acquired with a 10X objective at 20× final magnification. For multi-day acquisitions, organoids were kept in a stage-top incubator to maintain 37° C. , 5% CO2, and 95% relative humidity. Imaging was controlled with a Jupyter notebook that used a custom Python library (AcqPak) to manage experimental acquisitions and the Micro-Manager API for hardware control; all AcqPak software is freely available for download from Github (https://github.com/FordyceLab/AcqPack). Each position in a rastered acquisition was imaged with the following filter cubes and exposures: brightfield (Semrock BRFD-A-NTE-ZERO) at 1 ms, Cy5 (Semrock 49002) at 15 ms, and mCherry at 50 ms. Rasters were acquired every 2 hours.

### Initial Image Processing

All image processing was performed using custom Python libraries which are openly available for download from Github. To extract per-microwell information, we first stitched raw tiled images are first stitched into a single reconstructed image of the microwell array (ImageStitcher; https: //github. com/FordyceLab/ImageStitcher). The pixel locations of the array corners are used to rotate the image so the array edges are parallel with the image edges. Subarrays containing either 400 microwells (100 μm microwells) or 100 microwells (200 μm microwells) are extracted from the stitched array image using the corner pixel locations (Timecourse Processor; link), and image prediction for nuclear segmentation is performed with DeepCell on the subarrays (Wellception; link).

### Deepcell Model Training and Testing

To enable automated quantification of organoid growth from microwell images, we first trained a DeepCell deep learning model [ref] to predict fluorescent organoid nuclei. To construct the dataset used to train the model, individual microwell images of 114×114 pixels from experiment #1 were saved as. npz files, and each image was randomly assigned to either training data (80%), validation data (20%). Additional images from experiments #1- #3 were used as test data (306, 348, 258 images respectively). We used automated quality control to remove microwell images containing >8 cells after initial testing revealed images with higher cell numbers were challenging for annotators to label manually (data not shown), leaving a final dataset of 2711 training images, 542 validation images and 912 test images. The Caliban desktop module (26) was used to manually label individual nuclei in each image, and the annotations were saved to the. npz files. DeepCell-tf was then used to train a DeepCell deep learning model, with model weights stored in a hdf5 file, and performance was measured on the validation and test sets using the DeepCell-toolbox (25, 26).

To predict nuclei in microwell images, the organoid segmenter was initialized with trained Deepcell model weights stored in an hdf5 file. The segmenter took in input images and preprocessed them with histogram normalization using a kernel size of 32×32. Each macrowell was imaged in 9-15 subarrays, with each 2280×2280 pixel subarray containing 400 microwells of 114×114 pixels each (100 μm microwells) or 100 microwells of 224×224 pixels each (200 μm microwells). Each subsection was first rescaled by a factor of two before being passed into the model. DeepCell outputs were postprocessed with a watershed filter with a detection threshold of 0.25, a distance threshold of 0.1 and a minimum distance of 2.5. The model was run on a NVIDIA GPU. DeepCell nuclear predictions were saved as an hdf5 file containing all subarrays coming from a single microwell array. A. csv file was also generated containing summary statistics of the predictions for each timepoint and microwell (indexed by pixel location), including the predicted number of nuclei, area of each nucleus, the total area of the organoid, and centroid locations for each organoid, alongside pertinent metadata such as timepoint, microwell ID, mutant, and microwell pixel location.

### Quantification of Growth Rates Using Deepcell

To quantify organoid growth rates, we first used the extracted DeepCell predictions to identify microwells initially loaded with a 21 cells for which DeepCell identified ≥n+1 cells at the final timepoint in the time course. Total predicted organoid nuclear area was plotted as a function of time for up to 60 timepoints to include only timepoints with >8 cells. A model of exponential growth was then fit to the data, and parameters corresponding to initial area and growth rate as the percent change in area per day were extracted from the exponential model. To compare growth rate distributions across mutants and experiment, a one-way ANOVA with three variables was performed, and Bonferroni correction was used to account for multiple hypothesis testing.

### Polarity Measurements With Confocal Imaging

Confocal imaging to assess changes in apical-basal polarity was performed in microwells. Growth media was aspirated from plates, arrays were washed with 1X phosphate-buffered saline (PBS), the arrays were incubated in BD Cytofix/Cytoperm Fixation/Permeabilization solution for 30 min at 4° C., then cells were washed 3 times with 1X BD Perm/Wash buffer, with 10 minute incubations at room temperature between washes. Fixed cells were then stained with DAPI at 300 nM concentration diluted in PBS solution and Alexa Fluor 647 phalloidin at 165 nM concentration diluted in PBS solution, then incubated at room temperature in the dark for 1 hr. Cells were washed with PBS solution before imaging. Imaging was performed with a Leica SP5 upright multi-photon confocal microscope with a HCX APO L20×/1.00 water immersion objective. Organoid images were acquired in a z-stack with images taken every 10 μm. Representative single-plane images were extracted using Volocity software.

### Polarity Measurements With Live-cell Fluorescence Imaging

For experiments #1- #3, 1X SiR-actin dye (Cytoskeleton Inc.) and 1X verapamil were added to growth media before addition to macrowells at the start of the timecourse. For experiments #4- #6, growth media was aspirated from microwells at the final timepoint of the timecourse, and growth media supplemented with 1X SiR-actin dye and 1X verapamil was added to macrowells. This was done so in order to reduce the extrinsic effects introduced by the addition of the actin dye on the growth measurements of the organoids. Imaging, image processing, and individual microwell extraction were performed as described above. Blinded manual polarity classification was performed on a subset of images from the final timepoint of experiment #1, with 408 microwells classified for P53KO and 403 microwells for DKO. Organoids were classified as “normal”, “abnormal”, or “unknown”. To be classified as “abnormal”, organoids were required to display 2 of the following 3 characteristics: (1) disorganized actin signal not restricted to organoid lumens, (2) lack of a central lumen ringed with actin, and/or (3) the presence of multiple small, disorganized lumens. Aspiration of organoids with varying polarity was performed at the end of experiment #6, using the same classification requirements.

### Shallow WGS of Bulk Organoids

The P53KO and DKO organoids were harvested using TrypLE solution in a fashion similar to organoid passaging, prior to the start of experiments #4 and #6. The cells were then lysed and the nucleic acid was extracted using Qiagen Allprep DNA/RNA Mini Kit (Qiagen). Aliquots of DNA were sent to Novogene Co. for the construction of sequencing library and shallow WGS at 1X coverage. Sequencing reads were aligned to the hg38 human reference genome using BWA (52). Samtools (53) was used to convert the alignment files into bam format with indexes. The bam files were subsequently analyzed with QDNAseq (54) to infer copy-number variations using 50 kb read bin size and median normalization. The output was log2 transformed and visualized using a custom R script.

### Single Organoid Retrieval From Microwell

A syringe pump (Harvard Apparatus Pump 11 Elite) was used to drive a 1 ml syringe fitted with a blunt Luer probe tip. The syringe was attached to a 255 μm ID/510 μm OD PEEK tubing via a short 1 cm Tygon splint. The PEEK tubing was then threaded through a tightly-fitting 20 μl pipette tip such that approximately 0.5 cm extended past the tip. The splint and tip connections were secured with super glue. The tip was affixed to the microscope condenser z-stage using a 3D-printed liquid light guide holder (FIG. 4A). The syringe and tubing were primed with phosphate-buffered saline (PBS). The touch-down position of the tip was adjusted to the center of a live field of view under 4X magnification using thumbscrews on the condenser slot; this position was noted by drawing a reference box around the tip location in the Micro-Manager GUI. To pick an organoid of interest, the xy-stage was moved until the organoid was centered in the reference box. The tip was then lowered to the microwell surface using the condenser z-stage. The syringe pump was then used to withdraw the organoid into the tip; if necessary, adherent organoids were loosened by toggling withdraw/infuse on the syringe pump and/or by incubation with TrypLE for 5 minutes. The tip containing the aspirated organoid was then positioned just above an empty container (e.g. a well of a multiwell plate) and dispensed using the infuse button on the syringe pump into a 1.5 ml centrifuge tube.

A total of 20 organoids were retrieved from the DKO culture in microwell. Ten organoids had normal apical basal polarity as evidenced by the actin ring with a large lumen (FIG. 4B). Ten organoids had abnormal apical basal polarity which was defined as having two of the following three criteria: (1) disorganized actin signal not restricted to organoid lumens, (2) lack of a central lumen ringed with actin, and/or (3) the presence of multiple small, disorganized lumens.

### Dual ATAC/mRNA Library Preparations and Sequencing of Single Retrieved Organoids

Each single organoid retrieved from microwell was subjected to dual ATAC/mRNA sequencing library preparation following a recently published protocol that was designed for low input (55). Briefly, the organoid was spun down at 500 g for 5 minutes using a benchtop centrifuge. After removing supernatant, the organoid was then resuspended in a direct permeabilization/tagmentation mastermix (25 μl TD buffer (from Illumina Nextera XT DNA Library Prep Kit), 2.5 μl Tn5, 16.5 μl DPBS, 0.5 μl 1% digitonin, 0.5 μl 10% Tween-20, 2.5 μl RNase inhibitor and 2.5 μl nuclease-free water) without prior nuclei isolation. The reaction mixture was incubated in a 37° C. water bath for 30 minutes with occasional hand-pipetting.

At the end of incubation, 2.5 μl of stop buffer containing 10 mM EDTA and 0.5M lithium chloride was added to neutralize the permeabilization/tagmentation reaction. The tagmented cells were then lysed using 100 μl of Lysis/Binding Buffer from the Dynabeads mRNA Direct Micro Kit (Invitrogen). After lysis, 20 μl of pre-washed Dynabeads Oligo-dT beads were added to the lysate which was thereupon incubated at room temperature for 5 minutes to allow mRNA to anneal to the Oligo-dT. The beads with annealed mRNAs were separated from the supernatant using a magnetic rack. The supernatant which contained genomic DNA was transferred to a new tube for subsequent genomic DNA extraction using Qiagen MinElute PCR Purification Kit. Meanwhile, the beads-mRNA complex was resuspended in 20 μl of reverse transcription mastermix (without any primer) from Superscript IV First Strand cDNA Synthesis Kit (Invitrogen). The Oligo-dT on beads served as primers for the reverse transcription reaction. The reaction mix was incubated at 50° C. for 5 minutes then at 55° C. for an additional 10 minutes. The resulting cDNA/mRNA hybrid was thus covalently bound to the magnetic beads. The beads were washed twice with 100 μl ice-cold 10 mM Tris-HCl (pH 7), and resuspended in 5 μl of Tris-HCl. Sequencing library preparation of the cDNA was performed on beads using Nextera XT DNA Library Prep Kit where the reagent volumes were halved (keeping the reagent ratio consistent). Meanwhile, the previously tagmented ATAC-DNA was amplified using Q5 High-Fidelity 2X Mastermix (NEB) with universal i5 and indexed i7 adapters. PCR was performed with 18 cycles due to the low input nature of the samples. Both ATAC and cDNA libraries were cleaned using AMPure XP beads according to manufacturer's recommendations. The libraries were then sent to Novogene Co. for sequencing on the Illumina Novaseq platform with 75 bp paired-end reads.

### Bioinformatic Analysis of Atac-seq Sequencing Libraries

The raw sequencing reads were first processed with Trim-Galore to remove Illumina adapter sequences. After that, the Nextflow pipeline for atacseq (nf-core/atacseq) was used to process the sequencing trimmed reads in accordance with default parameters. Briefly, reads were aligned to hg38 human reference genome using BWA. The alignment files were further processed with Picard and Samtools to mark duplicate reads and to create bigWig files for downstream analysis and data visualization. Open chromatin peaks were called using MACS2 and annotated with HOMER. Due to low sample input, the number of PCR cycles used to generate the library was more than the recommended number, and this resulted in higher read duplication rate. As a result, the data was subjected to additional filters: 1) samples with less than 60000 deduplicated, uniquely mapped reads, and 2) samples with less than 500 total peaks were removed. The final dataset consisted of seven organoids each with normal and abnormal apical-basal polarity (FIG. 4C). The peak regions were then analyzed in DESeq2 to determine differential accessibility (56). The transcription factor footprinting analysis was performed using TOBIAS following default parameters (57).

### Bioinformatic Analysis of Mrna-seq Sequencing Libraries

The raw sequencing reads were first processed with Trim-Galore (58) to remove Illumina adapter sequences. The trimmed reads were then aligned to hg38 human reference transcriptome using STAR (59), and processed using RSEM to calculate gene expressions in each sample (60). The data was further examined with PCA analysis to remove outlier samples. In addition, the genes with zero readcount in more than or equal to 30% of the samples were removed from the differential analysis to ensure that the analysis was not skewed by missing data. DESeq2 was used to determine differential gene expression between the normal and abnormal groups.

### REFERENCES LISTING FOR REFERENCES CITED IN SPECIFICATION BY NUMBER

1. H. Clevers, Modeling Development and Disease with Organoids. Cell 165, 1586-1597 (2016).
2. G. Rossi, A. Manfrin, M. P. Lutolf, Progress and potential in organoid research. Nat Rev Genet 19, 671-687 (2018).
3 H. H. N. Yan et al., A Comprehensive Human Gastric Cancer Organoid Biobank Captures Tumor Subtype Heterogeneity and Enables Therapeutic Screening. Cell Stem Cell 23, 882-897.e811 (2018).
4. K. Han et al., CRISPR screens in cancer spheroids identify 3D growth-specific vulnerabilities. Nature 580, 136-141 (2020).
5. M. Kapałczyńska et al., 2D and 3D cell cultures—a comparison of different types of cancer cell cultures. Arch Med Sci 14, 910-919 (2018).
6. Y. H. Lo et al., A CRISPR/Cas9-Engineered. Cancer Discov 11, 1562-1581 (2021).
7. M. Matano et al., Modeling colorectal cancer using CRISPR-Cas9-mediated engineering of human intestinal organoids. Nat Med 21, 256-262 (2015).
8 B. E. Michels et al., Pooled In Vitro and In Vivo CRISPR-Cas9 Screening Identifies Tumor Suppressors in Human Colon Organoids. Cell Stem Cell 26, 782-792.e787 (2020).
9. K. Karlsson et al. (biorxiv, 2022).
10. P. Rawla, A. Barsouk, Epidemiology of gastric cancer: global trends, risk factors and prevention. Prz Gastroenterol 14, 26-38 (2019).
11. X. Qin, C. J. Tape, Deciphering Organoids: High-Dimensional Analysis of Biomimetic Cultures. Trends Biotechnol 39, 774-787 (2021).
12. N. Sachs, H. Clevers, Organoid cultures for the analysis of cancer phenotypes. Curr Opin Genet Dev 24, 68-73 (2014).
13. N. Brandenberg et al., High-throughput automated organoid culture via stem-cell aggregation in microcavity arrays. Nat Biomed Eng 4, 863-874 (2020).
14. J.A. Brassard, M. P. Lutolf, Engineering Stem Cell Self-organization to Build Better Organoids. Cell Stem Cell 24, 860-876 (2019).
15. X. Qin et al., Cell-type-specific signaling networks in heterocellular organoids. Nat Methods 17, 335-342 (2020).
16. W. Shin et al., Spatiotemporal Gradient and Instability of Wnt Induce Heterogeneous Growth and Differentiation of Human Intestinal Organoids. iScience 23, 101372 (2020).
17. M. Urbischek et al., Organoid culture media formulated with growth factors of defined cellular activity. Sci Rep 9, 6193 (2019).
18. P. Kakni et al., Intestinal Organoid Culture in Polymer Film-Based Microwell Arrays. Adv Biosyst 4, e2000126 (2020).
19. J. J. Tokar et al., Pairing Microwell Arrays with an Affordable, Semiautomated Single-Cell Aspirator for the Interrogation of Circulating Tumor Cell Heterogeneity. SLAS Technol 25, 162-176 (2020).
20. H.S. Shin, H. J. Hong, W. G. Koh, J. Y. Lim, Organotypic 3D Culture in Nanoscaffold Microwells Supports Salivary Gland Stem-Cell-Based Organization. ACS Biomater Sci Eng 4, 4311-4320 (2018).
21. A. Stern et al., The CellRaft AIR. SLAS Discov 27, 201-208 (2022).
22. C. Edlund et al., LIVECell-A large-scale dataset for label-free live cell segmentation. Nat Methods 18, 1038-1045 (2021).
23. G. I. Evan, K. H. Vousden, Proliferation, cell cycle and apoptosis in cancer. Nature 411, 342-348 (2001).
24. R. Halaoui, L. McCaffrey, Rewiring cell polarity signaling in cancer. Oncogene 34, 939-950 (2015).
25. D. A. Van Valen et al., Deep Learning Automates the Quantitative Analysis of Individual Cells in Live-Cell Imaging Experiments. PLoS Comput Biol 12, e1005177 (2016).
26. E. Moen et al. (biorxiv, 2019).
27. C. G. A. R. Network, Comprehensive molecular characterization of gastric adenocarcinoma. Nature 513, 202-209 (2014).
28. K. Wang et al., Exome sequencing identifies frequent mutation of ARID1A in molecular subtypes of gastric cancer Nat Genet 43, 1219-1223 (2011).
29. Y. L. Han et al., Cell swelling, softening and invasion in a three-dimensional breast cancer model. Nat Phys 16, 101-108 (2020).
30. Q. Yang et al., Cell fate coordinates mechano-osmotic forces in intestinal crypt formation. Nat Cell Biol 23, 733-744 (2021).
31. K. L. VanDussen, N. M. Sonnek, T. S. Stappenbeck, L-WRN conditioned medium for gastrointestinal epithelial stem cell culture shows replicable batch-to-batch activity levels across multiple research teams. Stem Cell Res 37, 101430 (2019).
32. J. Peng, A. Gassama-Diagne, Apicobasal polarity and Ras/Raf/MEK/ERK signalling in cancer. Gut 66, 986-987 (2017).
33. R. Baffa et al., Loss of FHIT expression in gastric carcinoma. Cancer Res 58, 4708-4714 (1998).
34. C. Huiping et al., High frequency of LOH, MSI and abnormal expression of FHIT in gastric cancer. Eur J Cancer 38, 728-735 (2002).
35. D. Boltin, Y. Niv, Mucins in Gastric Cancer—An Update. J Gastrointest Dig Syst 3, 15519 (2013).
36. F. Karasawa et al., Essential role of gastric gland mucin in preventing gastric cancer in mice. J Clin Invest 122, 923-934 (2012).
37. K. Song et al., Gastric mucin phenotype indicates aggressive biological behaviour in early differentiated gastric adenocarcinomas following endoscopic treatment. Diagn Pathol 16, 62 (2021).
38. S. M. Kim et al., Decreased Muc5AC expression is associated with poor prognosis in gastric cancer. Int J Cancer 134, 114-124 (2014).
39. S. Muthupalani et al., Muc5ac null mice are predisposed to spontaneous gastric antro-pyloric hyperplasia and adenomas coupled with attenuated H. pylori-induced corpus mucous metaplasia. Lab Invest 99, 1887-1905 (2019).
40. L. Tao, X. Wang, Q. Zhou, Long noncoding RNA SNHG16 promotes the tumorigenicity of cervical cancer cells by recruiting transcriptional factor SPI1 to upregulate PARP9. Cell Biol Int 44, 773-784 (2020).
41. P. Rimmelé et al., Spi-1 /PU.1 oncogene accelerates DNA replication fork elongation and promotes genetic instability in the absence of DNA breakage. Cancer Res 70, 6757-6766 (2010).
42. Q. Van Thillo et al., Oncogenic cooperation between TCF7-SPI1 and NRAS(G12D) requires ß-catenin activity to drive T-cell acute lymphoblastic leukemia. Nat Commun 12, 4164 (2021).
43. J. Liu et al., The PP2A-Aß gene is regulated by multiple transcriptional factors including Ets-1, SP1/SP3, and RXRα/β. Curr Mol Med 12, 982-994 (2012).
44. K. Rust, A. Wodarz, Transcriptional Control of Apical-Basal Polarity Regulators. Int J Mol Sci 22,(2021).
45. E. Hedrick, Y. Cheng, U. H. Jin, K. Kim, S. Safe, Specificity protein (Sp) transcription factors Sp1, Sp3 and Sp4 are non-oncogene addiction genes in cancer cells. Oncotarget 7, 22245-22256 (2016).
46. S. Safe, M. Abdelrahim, Sp transcription factor family and its role in cancer. Eur J Cancer 41, 2438-2448 (2005).
47. M. Fomicheva, E. M. Tross, I. G. Macara, Polarity proteins in oncogenesis. Curr Opin Cell Biol 62, 26-30 (2020).
48. Y. Jossin et al., Llgl1 Connects Cell Polarity with Cell-Cell Adhesion in Embryonic Neural Stem Cells. Dev Cell 41, 481-495.e485 (2017).
49. J. Weber, C. J. Braun, D. Saur, R. Rad, In vivo functional screening for systems-level integrative cancer genomics. Nat Rev Cancer 20, 573-593 (2020).
50. X. Li et al., Site-specific labeling of annexin V with F-18 for apoptosis imaging. Bioconjug Chem 19, 1684-1688 (2008).
51. A. C. F. Bolhaqueiro et al., Ongoing chromosomal instability and karyotype evolution in human colorectal cancer organoids. Nat Genet 51, 824-834 (2019).
52. H. Li, R. Durbin, Fast and accurate short read alignment with Burrows-Wheeler transform. Bioinformatics 25, 1754-1760 (2009).
53. H. Li et al., The Sequence Alignment/Map format and SAMtools. Bioinformatics 25, 2078-2079, 2009
54. I. Scheinin et al., DNA copy number analysis of fresh and formalin-fixed specimens by shallow whole-genome sequencing with identification and exclusion of problematic regions in the genome assembly. Genome Res 24, 2022-2032 (2014).
55. R. Li, S. A. Grimm, P. A. Wade, A simple and robust method for simultaneous dual-omics profiling with limited numbers of cells. Cell Rep Methods 1, (2021).
56. M. I. Love, W. Huber, S. Anders, Moderated estimation of fold change and dispersion for RNA-seq data with DESeq2. Genome Biol 15, 550 (2014).
57. M. Bentsen et al., ATAC-seq footprinting unravels kinetics of transcription factor binding during zygotic genome activation. Nat Commun 11, 4267 (2020).
58. F. Krueger, F. James, P. Ewels, E. Afyounian, B. Schuster-Boeckler. (2021).
59. A. Dobin et al., STAR: ultrafast universal RNA-seq aligner. Bioinformatics 29, 15-21 (2013).
60. B. Li, C. N. Dewey, RSEM: accurate transcript quantification from RNA-Seq data with or without a reference genome. BMC Bioinformatics 12, 323 (2011).

1. A method of monitoring organoid development in parallel for a plurality of organoids, each arising from one or more organoid progenitor cells, said method comprising:
(i) distributing organoid progenitor cells from a population of organoid progenitor cells into array compartments to provide an open planar array comprising a plurality of organoid growth compartments in which each organoid growth compartment comprises one or more organoid progenitor cells;
(ii) incubating cells in the organoid growth compartments of the open planar array under growth conditions in which the one or more organoid progenitor cells in each organoid growth compartment divides;
(iii) imaging compartments of the open planar array at multiple time intervals;
(iv) analyzing at least one phenotypic parameter of multi-cell structures growing in at least a portion of the organoid growth compartments imaged in step (iii).

2. The method of claim 1, wherein distribution of organoid progenitor cells into the open planar array provides a plurality of organoid growth compartments, each comprising a single organoid progenitor cell.

3. The method of claim 1, wherein organoids in each grow compartment of the open planar array are cultured under the same conditions.

4. The method of claim 1, wherein the population of organoid progenitor cells is genetically distinct across the population.

5. The method of claim 1, wherein (iii) and (iv) occur simultaneously.

6. The method of claim 1, further comprising distributing additional co-culture cells into at least a portion of the organoid growth compartments containing organoid progenitor cells or a multi-cellular structure arising from the organoid progenitor cells.

7. The method of claim 6, wherein the co-culture cells are immune system cells, optionally wherein the immune system cells are T cells, dendritic cells, macrophages, or natural killer cells.

8. (canceled)

9. The method of claim 1, wherein the at least one phenotypic parameter is rate of cell division, cell polarity, or cell migration.

10. The method of claim 1, wherein step (iv) comprises detecting a signal from a cellular molecule labeled with a detectable label, optionally wherein the detectable label is a fluorescent label.

11. (canceled)

12. The method of claim 10, wherein the population of organoid stem cells is genetically modified to introduce the cellular molecule into cells.

13. The method of claim 10, wherein the fluorescent label is attached to an agent that specifically binds the molecule.

14. The method of claim 1, wherein the population of organoid progenitor cells comprises epithelial cells.

15. The method of claim 1, wherein the population of organoid progenitor cells comprises cells having a mutation at one or more genetic loci associated with a cancer and/or cells having mutations at two or more genetic loci associated with the cancer, optionally wherein the cancer is gastric cancer, colorectal cancer, pancreatic cancer, breast cancer, ovarian cancer, uterine cancer, prostate cancer, bladder cancer, liver cancer, lung cancer, esophageal cancer, head and neck cancer, or renal cancer and/or one or more mutations at genetic loci associated with cancer are introduced into the cell by gene editing.

16.-18. (canceled)

19. The method of claim 1, wherein:
the compartments are microwells;
step (i) comprises loading cells using centrifugation; or
imaging occurs at least once a day following distribution of single organoid stems cells into compartments.

20-21. (canceled)

22. The method of claim 1 wherein the method further comprises retrieving cells from at least a portion of the organoid growth compartments for genetic analysis, optionally wherein genetic analysis comprises RNA sequence, ATAC sequence analysis, whole genome sequencing, or methylation profiling.

23. The method of claim 22, wherein retrieving cells comprises aspirating cells.

24. (canceled)

25. A platform for monitoring organoid growth comprising (i) an open planar array comprising at least 100 organoid growth compartments, wherein each organoid growth compartment is a microwell comprising a three dimensional multi-cellular structure arising from one or more organ progenitor cells and (ii) an imaging system.

26. The platform of claim 25, wherein each of the three dimensional multi-cellular structures in a plurality of microwells comprises cells comprising a molecule labeled with a fluorescent label.

27. The platform of claim 26, wherein the fluorescent label is attached to an agent that specifically binds the molecule.

28. The platform of claim 25, wherein each three dimensional multi-cellular structure in a plurality of microwells comprises epithelial cells.

29. The platform of claim 25, wherein each three dimensional multi-cellular structure in a plurality of microwells comprises cells having a mutation at one or more genetic loci associated with cancer.

30. The platform of claim 29, wherein the three dimensional multi-cellular structure arose from a single cell that was genome edited to comprise the mutation; and/ or the cancer is gastric cancer, colorectal cancer, pancreatic cancer, breast cancer, ovarian cancer, uterine cancer, prostate cancer, bladder cancer, liver cancer, lung cancer, esophageal cancer, head and neck cancer, brain cancer, or renal cancer.

31. (canceled)
